(19) United States
(12) Patent Application Publication
(10) Pub. No.: US 2024/0407773 A1
(43) Pub. Date: Dec. 12, 2024

(54) STIFFER CORE MATERIAL FOR RF PERFORATION DEVICE

(71) Applicant: Boston Scientific Scimed, Inc., Maple Grove, MN (US)

(72) Inventors: Brice Lee Shireman, Maple Grove, MN (US); Spencer Fodness-Bondhus, Columbia Heights, MN (US)

(21) Appl. No.: 18/737,584

(22) Filed: Jun. 7, 2024

Related U.S. Application Data
(60) Provisional application No. 63/507,172, filed on Jun. 9, 2023.

(51) Int. Cl. A61B 17/00

(52) U.S. Cl. CPC A61B 17/00234; A61B 2017/00247

(57) ABSTRACT

A perforation device for transseptal access system is disclosed. The perforation device includes an electrically conductive core comprising a proximal portion having a proximal portion length, and a distal portion having a distal portion length. The proximal portion is formed from a first material having a first modulus of elasticity, and the distal portion is formed from a second material having a second modulus of elasticity that is lower than the first modulus of elasticity. The perforation device further includes an exposed electrically conductive functional tip at a distal end of the core. Finally, the perforation device also includes an insulation layer over a portion of the core.

### CROSS REFERENCE TO RELATED APPLICATION

The present application claims priority to U.S. Provisional Patent Application Ser. No. 63/507,172, filed Jun. 9, 2023, the entire disclosure of which is incorporated herein in its entirety.

### TECHNICAL FIELD

The present invention relates generally to methods and devices used to deliver energy within the body of a patient. More specifically, the present invention is concerned with a radiofrequency perforation apparatus.

### BACKGROUND

Devices currently exist for creating a puncture, channel, or perforation within a tissue located in a body of a patient. One such device is the Brockenbrough™ Needle, which is commonly used to puncture the atrial septum of the heart. This device is a stiff elongate needle, which is structured such that it may be introduced into a body of the patient via the femoral vein and directed towards the heart. This device relies on the use of mechanical force to drive the sharp tip through the septum.

Alternatively, radiofrequency perforation apparatuses have been developed, whereby the septal perforation is accomplished by the application of focused radiofrequency energy to the septal tissue via an electrode at the distal end of a relatively thin conductive probe.

Against this background, there exists a continuing need in the industry to provide safe and improved radiofrequency perforation devices and methods. An object of the present invention is therefore to provide such a radiofrequency perforation apparatus.

### SUMMARY

Example 1 is a perforation device for transseptal access system, the perforation device includes an electrically conductive core comprising a proximal portion having a proximal portion length, and a distal portion having a distal portion length, wherein the proximal portion is formed from a first material having a first modulus of elasticity, and the distal portion is formed from a second material having a second modulus of elasticity that is lower than the first modulus of elasticity. The perforation device also includes an exposed electrically conductive functional tip at a distal end of the core. The perforation device further includes an insulation layer over a portion of the core.

Example 2 is the perforation device of Example 1 wherein the proximal portion has a first diameter and the distal portion has a second diameter that is greater than the first diameter.

Example 3 is the perforation device of Example 2 wherein the insulation layer has a first thickness over the proximal portion of the core, and the insulation layer has a second thickness over the distal portion of the core, wherein the first thickness is greater than the second thickness.

Example 4 is the perforation device of Example 1 wherein the proximal and distal portions of the core have substantially the same diameter.

Example 5 is the perforation device of any of Examples 1-3 wherein the perforation device is substantially isodiametric along substantially the entire length of the core.

Example 6 is the perforation device of any of Examples 1-5 wherein the proximal and distal portions of the core are mechanically attached together at a joint.

Example 7 is the perforation device of any of Examples 1-6 wherein the first material is a tungsten copper alloy or a molybdenum copper alloy.

Example 8 is the perforation device of any of Examples 1-7 wherein the second material is a copper cladded stainless steel or a platinum core stainless steel.

Example 9 is the perforation device of any of Examples 1-6 wherein the proximal and distal portions of the core are attached by welding, brazing, soldering, bonding, and the like.

Example 10 is the perforation device of Example 6 wherein the proximal portion includes a shank and the distal portion includes a socket.

Example 11 is the perforation device of Example 10 wherein the shank is received within the socket to provide a connection between the proximal portion and the distal portion.

Example 12 is the perforation device of any of Examples 1-11 wherein the proximal portion and the distal portion have substantially equal bending stiffnesses.

Example 13 is the perforation device of any of Examples 1-11 wherein at least part of the proximal portion is configured to be operable as a support rail for delivery of a secondary device over the perforation device.

Example 14 is the perforation device of any of Examples 1-12 wherein the functional tip has a tip diameter that is substantially equal to a diameter defined by the electrical insulation at a distal end of the core.

Example 15 is the perforation device of any of Examples 1-14 wherein the functional tip has a tip diameter that is substantially equal to a diameter defined by the electrical insulation at a distal end of the core.

Example 16 is a perforation device for transseptal access system, the perforation device includes an electrically conductive core comprising a proximal portion having a proximal portion length, and a distal portion having a distal portion length, wherein the proximal portion is formed from a first material having a first modulus of elasticity, and the distal portion is formed from a second material having a second modulus of elasticity that is lower than the first modulus of elasticity. The perforation device further includes an exposed electrically conductive functional tip at a distal end of the core.

Example 17 is the perforation device of Example 16 wherein the perforation device further includes an insulation layer over a portion of the core.

Example 18 is the perforation device of Example 16 wherein the proximal portion has a first diameter and the distal portion has a second diameter that is greater than the first diameter.

Example 19 is the perforation device of Example 17 wherein the insulation layer has a first thickness over the proximal portion of the core, and the insulation layer has a second thickness over the distal portion of the core, wherein the first thickness is greater than the second thickness.

Example 20 is the perforation device of Example 16 wherein the proximal and distal portions of the core have substantially the same diameter.

Example 21 is the perforation device of Example 16 wherein the perforation device is substantially isodiametric along substantially the entire length of the core.

Example 22 is the perforation device of Example 16 wherein the proximal and distal portions of the core are mechanically attached together at a joint, and wherein the proximal and distal portions of the core are attached by welding, brazing, soldering, bonding, and the like.

Example 23 is the perforation device of Example 16 wherein the first material is a tungsten copper alloy or a molybdenum copper alloy.

Example 24 is the perforation device of Example 16 wherein the second material is a copper cladded stainless steel or a platinum core stainless steel.

Example 25 is the perforation device of Example 16 wherein the proximal portion and the distal portion have substantially equal bending stiffnesses.

Example 26 is a perforation device for transseptal access system, the perforation device includes an electrically conductive core comprising a proximal portion having a proximal portion length, and a distal portion having a distal portion length, wherein the proximal portion is formed from a first material having a first modulus of elasticity, and the distal portion is formed from a second material having a second modulus of elasticity that is lower than the first modulus of elasticity. The perforation device also includes an exposed electrically conductive functional tip at a distal end of the core. The perforation device further includes an insulation layer over a portion of the core.

Example 27 is the perforation device of Example 26 wherein the proximal portion has a first diameter and the distal portion has a second diameter that is greater than the first diameter.

Example 28 is the perforation device of Example 26 wherein the insulation layer has a first thickness over the proximal portion of the core, and the insulation layer has a second thickness over the distal portion of the core, wherein the first thickness is greater than the second thickness.

Example 29 is the perforation device of Example 26 wherein the proximal and distal portions of the core have substantially the same diameter.

Example 30 is the perforation device of Example 26 wherein the perforation device is substantially isodiametric along substantially the entire length of the core.

Example 31 is the perforation device of Example 26 wherein the proximal and distal portions of the core are mechanically attached together at a joint, and wherein the proximal and distal portions of the core are attached by welding, brazing, soldering, bonding, and the like.

Example 32 is the perforation device of Example 26 wherein the first material is a tungsten copper alloy or a molybdenum copper alloy.

Example 33 is the perforation device of Example 26 wherein the second material is a copper cladded stainless steel or a platinum core stainless steel.

Example 34 is the perforation device of Example 26 wherein the perforation device is configured to be operatively coupled to a radiofrequency generator for delivery of radiofrequency energy to the functional tip.

Example 35 is a method of making a perforation device for transseptal access system, the method includes providing an electrically conductive core comprising a proximal portion having a proximal portion length, and a distal portion having a distal portion length, wherein the proximal portion is formed from a first material having a first modulus of elasticity, and the distal portion is formed from a second material having a second modulus of elasticity that is lower than the first modulus of elasticity. The method of making a perforation device also includes securing an exposed electrically conductive functional tip at a distal end of the core. The method of making a perforation device further includes securing an insulation layer over a portion of the core.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the disclosure.

FIG. 2 is a schematic illustration of a radiofrequency perforation device for use in the transseptal access system of FIGS. 1A-1C, according to embodiments of the present disclosure.

FIGS. 3A-3C are schematic cross-sectional illustrations of an electrically conductive core of the radiofrequency perforation device of FIG. 2, according to embodiments of the present disclosure.

FIGS. 4A-4C are schematic cross-sectional illustrations of a portion of the radiofrequency perforation device of FIGS. 3A-3C, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

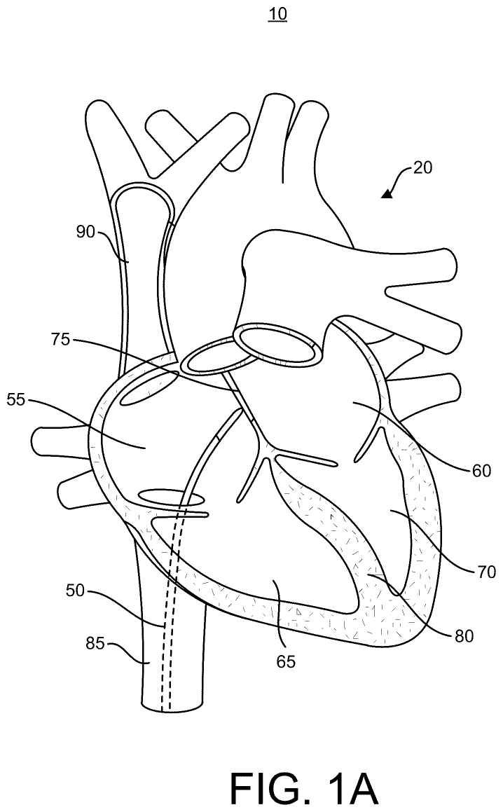

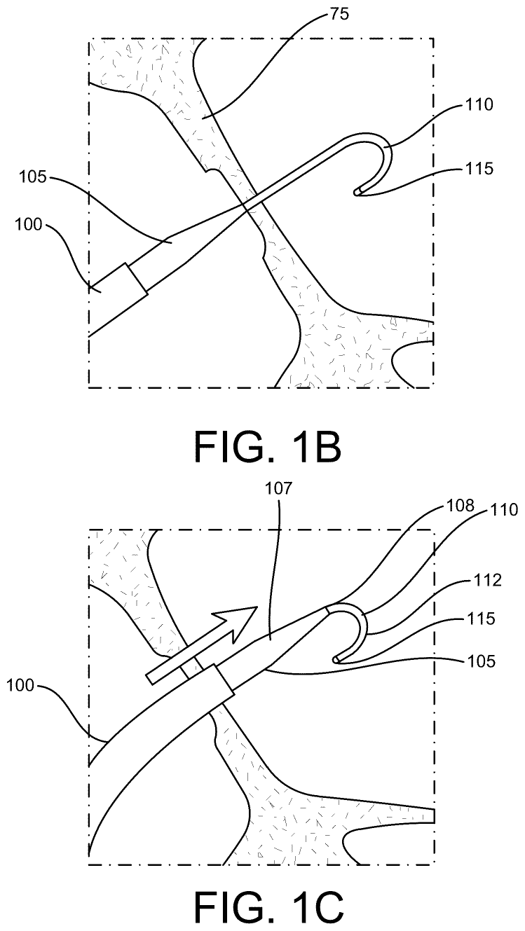

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75, and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110 having distal end portion 112 terminating in a tip electrode 115. As shown, in the assembled use state illustrated in FIGS. 1B and 1C, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown but explained in greater detail herein) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the superior vena cava, the guidewire is removed from the body, and the sheath 100 and the dilator 105 are advanced so that their distal ends are positioned in the right atrium 55. The RF perforation device 110 described can then be introduced into the dilator 105, and advanced toward the heart 20.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 s and about 5 s.

With the tip electrode 115 of the RF perforation device 110 having crossed the atrial septum 75, the dilator 105 can be advanced forward, with the tapered distal tip portion 107 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape or pigtail shape. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60, and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device into the left atrium 60. The anchoring function of the pre-formed distal end portion 112 inhibits unintended retraction of the distal end portion 112, and corresponding loss of access to the perforated site on the atrial septum 75, during such withdrawal.

The present disclosure describes novel devices and methods for providing transseptal access to the left atrium 60 using radiofrequency energy. As will be explained in greater detail herein, the embodiments of the present disclosure describe RF perforation devices providing enhanced protection to the user from exposure to radiofrequency energy without adversely affecting the performance of the device. Additionally, in embodiments, the RF perforation devices of the present disclosure can provide higher electrical energy while also efficiently transferring the generated energy to the target tissue in order to minimize procedure times.

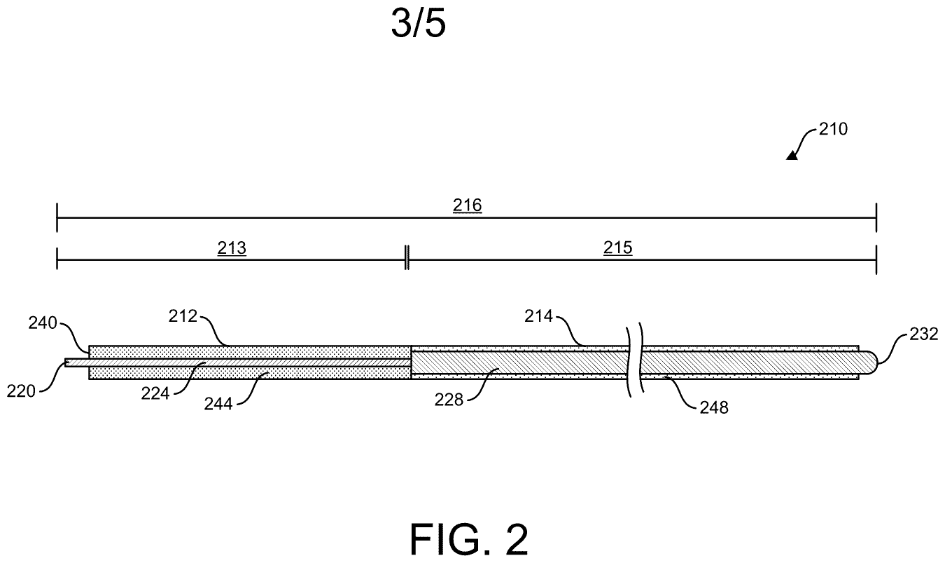

FIG. 2 is a schematic cross-sectional illustration of a radiofrequency perforation device 210 for use in the transseptal access system of FIGS. 1A-1C, according to embodiments of the present disclosure. As can be seen from FIG. 2, the RF perforation device 210 has a proximal portion 212 having a proximal portion length 213, and a distal portion 214 extending from the proximal portion 212 and having a distal portion length 215. The proximal and distal portion lengths 212, 214 together define an overall perforation device length 216. As will be appreciated, the perforation device length 216 is greater than the length of the dilator (not shown in FIG. 2, but can be seen in FIGS. 1A-1C) with which it is used, such that part of the proximal portion 212 of the RF perforation device 210 extends proximally of the dilator when the distal portion 214 extends distally of the dilator, thus allowing the proximal portion 212 to be manipulated by the user as needed. In embodiments, the proximal portion 212 of the RF perforation device 210 has an electrically conductive core 220 having a core proximal portion 224, a core distal portion 228 and a functional tip 232 (e.g., a tip electrode as described above in connection with FIGS. 1A-1C) at the distal extremity of the distal portion 228.

As further shown, the RF perforation device 210 has an electrical insulation layer 240 disposed over a majority of the core 220. The insulation layer 240 includes an insulation proximal portion 244 disposed over the core proximal portion 224, and an insulation distal portion 248 disposed over the core distal portion 228. As further shown, in the illustrated embodiment, a segment of the core proximal portion 224 is not covered by the insulation proximal portion 244 and thus is electrically exposed to facilitate operatively coupling the RF perforation device 210 to an external energy source (i.e., an RF generator) via a connector cable (not shown). The skilled artisan will recognize that the particular technique for facilitating such coupling is shown schematically in FIG. 2 for ease of illustration, and that a number of suitable techniques for electrically coupling the RF perforation device 210 to an energy source can be employed within the scope of the disclosure.

As can be seen in FIG. 2, the core proximal portion 224 coincides with and generally defines the proximal portion 212 of the RF perforation device 210, and the core distal portion 228 and the functional tip 232 in combination define the distal portion 214 of the RF perforation device 210. As further shown, in the illustrated embodiment the insulation distal portion 248 terminates proximally of the functional tip 232 so as to expose the functional tip 232 to target tissue for performing a septal perforation procedure, although in embodiments the functional tip 232 may be recessed within the insulation distal portion 248. The disposition of the insulation proximal portion 244 over the core proximal portion 224 permits the user to directly handle the proximal portion 212 when the RF perforation device 210 is energized.

In general, the insulation layer 240 can be made from any number of biocompatible materials having suitable dielectric properties. One exemplary class of materials for construction of the insulation layer can include various grades of polytetrafluoroethylene (PTFE), fluorinated ethylene propylene (FEP), polyetheretherketone (PEEK), polyethylene terephthalate (PET), polyimide, among others. The core 220 is electrically conductive and is capable of transferring radiofrequency energy supplied by an external RF generator (not shown) to the functional tip 232 for subsequent delivery to the target tissue in a transseptal crossing procedure, as described above. In general, the core 220 is constructed in a manner to provide desired operational functionality, e.g., energy transfer, handleability, tactile feel, flexural rigidity, columnar strength, and the like.

In embodiments, the core proximal and core distal portions 224, 228 are made from different materials having different moduli of elasticity. In this manner, the overall mechanical properties of the respective portions of the RF perforation device 210 can be tailored according to the particular clinical needs therefor, as will be further explained below. In embodiments, the core proximal portion 224 is made of a material having a higher modulus of elasticity than the material used for the core distal portion 228.

For ease of illustration, the RF perforation device 210 is shown in FIG. 2 as being substantially isodiametric along its entire length, but this is not critical. Rather, the diameter of the proximal and/or distal portions 212, 214 may vary along their respective lengths depending on the particular functional requirements for a given procedure. For example, portions of the core proximal portion 212 may have an increased diameter relative to other portions to, among other things, provide enhanced manipulability by the user and/or to delimit the degree to which the RF perforation device 210 can be advanced distally within the dilator. Still additionally, the distal-most regions of the distal portion 214 may have a diameter that is greater or less than the more proximal regions so as to tailor the stiffness and columnar strength of the respective regions, as will be explained in greater detail herein.

Furthermore, the RF perforation device 210 may include additional features to enhance its functionality and usability. By way of example, the RF perforation device 210 may include imaging markers (e.g., radiopaque or echogenic structures) at selected locations to enhance the visibility of the devices under imaging modalities. As another example, fiducial markers may be included along the proximal portion 212 of the RF perforation device 210 to provide the user with a visual indication of the relative positions of the RF perforation device 210 and the dilator. Still other value-added features may be utilized to enhance the usability of the system 50 of FIGS. 1A-1C.

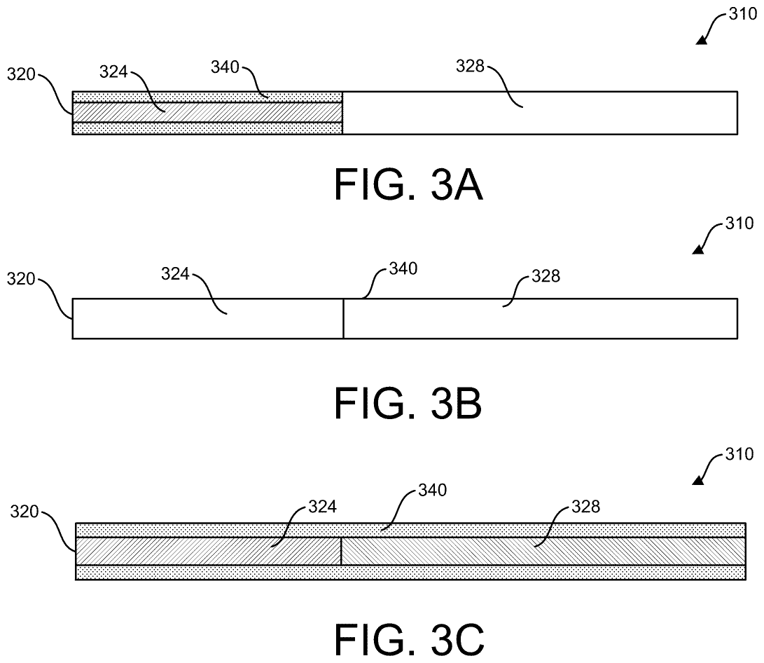

FIGS. 3A-3C are schematic cross-sectional illustrations of an electrically conductive core 320 of an RF perforation device 310, according to embodiments of the present disclosure. The RF perforation device 310 may be substantially structurally and functionally identical to the RF perforation device 210, except as described in connection with FIGS. 3A-3C. As shown in FIGS. 3A-3C, the conductive core 320 includes a core proximal portion 324 and a core distal portion 328, corresponding with the core proximal and core distal portions 224, 228 of the RF perforation device 210 of FIG. 2. In embodiments, the RF perforation device 310 further includes a functional tip (e.g., a tip electrode as described above in connection with FIGS. 1A-1C) at the distal extremity of the core distal portion 328. In further embodiments, the RF perforation device 310 may include an insulation layer 340 with varying levels of thickness on the core proximal portion and/or the core distal portion, as will be explained below. In general, the insulation layer 340 can be made from any number of biocompatible materials having suitable dielectric properties. One exemplary class of materials for construction of the insulation layer can include various grades of polytetrafluoroethylene (PTFE), fluorinated ethylene propylene (FEP), polyetheretherketone (PEEK), polyethylene terephthalate (PET), polyimide, among others. Additionally, in embodiments, a joint is located at the junction of the core proximal and core distal portions 324, 328, as will be discussed in greater detail herein.

In embodiments, the core proximal and core distal portions are made from different materials having different moduli of elasticity. In embodiments, the core distal portion 328 is made of copper cladded stainless steel, platinum core stainless steel, and the like. In embodiments, stainless steel provides good strength and stiffness for a distal section of a guidewire. In embodiments, the core proximal portion 324 is made of a material having a higher modulus of elasticity than the material used for the core distal portion 328. The higher modulus proximal core allows the RF perforation device guidewire to have more rail support. Furthermore, the core proximal portion being made of a material having a higher modulus of elasticity would also allow for thicker electrical insulation around the core proximal portion 324, providing more safety to the user. In one embodiment, the core proximal portion 324 is made of tungsten copper alloy, molybdenum copper alloy, and the like, instead of stainless steel. In embodiments, tungsten alloy and molybdenum alloy have a higher modulus than stainless steel providing additional stiffness.

A stiffer core material, like tungsten copper alloy or molybdenum copper alloy for the core proximal portion 324, would allow the RF perforation device 310 to have the same stiffness profile as the core distal portion but with a smaller proximal diameter core. This allows more space radially to attach a thicker insulative wall material, which further provides more safety to the user and allows the user to use higher power radiofrequency energy for the selected procedure. In further embodiments, the diameter of the core proximal and core distal portions 324, 328 of FIGS. 3A-3C may vary along their respective lengths depending on the particular functional requirements for a given procedure. In embodiments, portions of the core proximal portion 324 may have a smaller diameter relative to the core distal portion 328 or the same diameter as the core distal portion so as to tailor the stiffness and columnar strength of the respective regions.

In embodiments, as shown in FIG. 3A, the higher modulus core proximal portion 324 is smaller in diameter than the stainless steel core distal portion 328. This allows for the stiffness of the core distal portion 328 and the stiffness of the core proximal portion 324 to be closely matched while allowing space for a thicker insulative layer 340 around the core proximal portion 324. The insulation layer 340 may provide additional electrical insulation for higher radiofrequency voltage application. In embodiments, the core distal portion 328 may also include a thin insulative layer. In other embodiments, as shown in FIG. 3B, the higher modulus tungsten core proximal portion 324 may be slightly smaller or equal in diameter to the stainless steel core distal portion 328. In embodiments, this allows for a thin insulative sleeve 340 to be layered around the core proximal portion 324 and the core distal portion 328 which then permits the RF perforation device 310 to provide additional support for delivery of adjunct devices, possible delivery of additional electrical insulation for higher radiofrequency voltage applications, among other benefits. In further embodiments, as shown in FIG. 3C, both the core proximal portion 324 and the core distal portion 328 can be made from of a material having a higher modulus of elasticity, preferably tungsten copper alloy, molybdenum copper alloy, and the like. If the higher modulus core is used for the entire length of the RF perforation device 310, a thicker insulative layer 340 may be used over the full length of the core 320. This would permit higher radiofrequency energy transmission through the RF perforation device 310 for the transseptal access system 50 of FIGS. 1A-1C.

In such embodiments, the core proximal portion 324 may have a diameter of between about 0.03 and 1 millimeters and, in some embodiments, between about 0.038 to 0.965 millimeters, and the core distal portion 328 may have a maximum diameter of about 0.038 to 0.965 millimeters. As such, as illustrated in FIGS. 3A-3C, the insulation around the core proximal portion 324 may have a greater layer thickness than the insulation around the core distal portion 328 while still maintaining a substantially isodiametric configuration, while maintaining substantially constant mechanical properties such as flexural rigidity and columnar strength from the proximal end of the RF perforation device 310 through at least a portion of the distal portion thereof. In the illustrated embodiment, the insulation of the proximal portion 324 may have a wall thickness of between about 0.05 and 0.5 millimeters and, in some embodiments, between about 0.051-0.406 millimeters. In various embodiments, the insulation of the distal most portion of the distal portion 328 may have a wall thickness of between about 0.1 and 0.5 millimeter and, in some embodiments, between about 0.108-0.463 millimeters, while the proximal portion of the distal portion 328 may have a wall thickness of between about 0.05 and 0.4 millimeters and, in some embodiments, between about 0.051 to 0.368 millimeters. The relatively thicker insulation around the core proximal portion can operate to minimize user exposure to RF energy while handling the proximal portion of an energized RF perforation device 310.

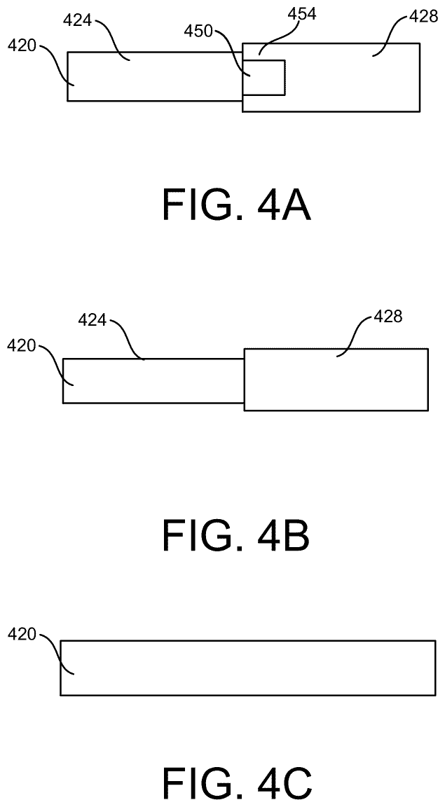

FIGS. 4A-4C are schematic cross-sectional illustrations of a portion of a core 420 corresponding with the core 320 of the RF perforation device 310 of FIGS. 3A-3C, specifically, in the region of the joint located at the junction of a core proximal portion and a core distal portion, according to embodiments of the present disclosure. As shown, the core 420 includes a core proximal portion 424 and a core distal portion 428. In embodiments, the junction between the core proximal portion 424 and the core distal portion 428 can be joined by a mechanical joining process, e.g., laser welding, friction welding, brazing, soldering, adhesive bonding and the like.

In embodiments, as illustrated in FIG. 4A, the core proximal portion 424 includes a shank 450 having a slightly smaller diameter than the adjacent portion of the core proximal portion 424. Furthermore, the core distal portion 428 includes a socket 454 extending axially. As shown in FIG. 4A, the shank 450 is received within the socket 454 to provide a robust mechanical connection between the core proximal portion 424 and the core distal portion 428, also known as a male-female connection. In other embodiments, as illustrated in FIG. 4B, the core proximal and core distal portions 424, 428 can be joined via a butt joint, i.e., the mating shank 450 and socket 454 may be omitted, and one of the aforementioned joining processes can be employed to further reinforce the joint. In still other embodiments, as illustrated in FIG. 4C, the wire may have a unibody core 420 with no injunctions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A perforation device for transseptal access system, the perforation device comprising:
an electrically conductive core comprising a proximal portion having a proximal portion length, and a distal portion having a distal portion length, wherein the proximal portion is formed from a first material having a first modulus of elasticity, and the distal portion is formed from a second material having a second modulus of elasticity that is lower than the first modulus of elasticity; and
an exposed electrically conductive functional tip at a distal end of the core.

2. The perforation device of claim 1, wherein the perforation device further includes an insulation layer over a portion of the core.

3. The perforation device of claim 1, wherein the proximal portion has a first diameter and the distal portion has a second diameter that is greater than the first diameter.

4. The perforation device of claim 2, wherein the insulation layer has a first thickness over the proximal portion of the core, and the insulation layer has a second thickness over the distal portion of the core, wherein the first thickness is greater than the second thickness.

5. The perforation device of claim 1, wherein the proximal and distal portions of the core have substantially the same diameter.

6. The perforation device of claim 1, wherein the perforation device is substantially isodiametric along substantially the entire length of the core.

7. The perforation device of claim 1, wherein the proximal and distal portions of the core are mechanically attached together at a joint, and wherein the proximal and distal portions of the core are attached by welding, brazing, soldering, bonding, and the like.

8. The perforation device of claim 1, wherein the first material is a tungsten copper alloy or a molybdenum copper alloy.

9. The perforation device of claim 1, wherein the second material is a copper cladded stainless steel or a platinum core stainless steel.

10. The perforation device of claim 1, wherein the proximal portion and the distal portion have substantially equal bending stiffnesses.

11. A perforation device for transseptal access system, the perforation device comprising:
an electrically conductive core comprising a proximal portion having a proximal portion length, and a distal portion having a distal portion length, wherein the proximal portion is formed from a first material having a first modulus of elasticity, and the distal portion is formed from a second material having a second modulus of elasticity that is lower than the first modulus of elasticity;
an exposed electrically conductive functional tip at a distal end of the core; and
an insulation layer over a portion of the core.

12. The perforation device of claim 11, wherein the proximal portion has a first diameter and the distal portion has a second diameter that is greater than the first diameter.

13. The perforation device of claim 11, wherein the insulation layer has a first thickness over the proximal portion of the core, and the insulation layer has a second thickness over the distal portion of the core, wherein the first thickness is greater than the second thickness.

14. The perforation device of claim 11, wherein the proximal and distal portions of the core have substantially the same diameter.

15. The perforation device of claim 11, wherein the perforation device is substantially isodiametric along substantially the entire length of the core.

16. The perforation device of claim 11, wherein the proximal and distal portions of the core are mechanically attached together at a joint, and wherein the proximal and distal portions of the core are attached by welding, brazing, soldering, bonding, and the like.

17. The perforation device of claim 11, wherein the first material is a tungsten copper alloy or a molybdenum copper alloy.

18. The perforation device of claim 11, wherein the second material is a copper cladded stainless steel or a platinum core stainless steel.

19. The perforation device of claim 11, wherein the perforation device is configured to be operatively coupled to a radiofrequency generator for delivery of radiofrequency energy to the functional tip.

20. A method of making a perforation device for transseptal access system, the method comprising:
providing an electrically conductive core comprising a proximal portion having a proximal portion length, and a distal portion having a distal portion length, wherein the proximal portion is formed from a first material having a first modulus of elasticity, and the distal portion is formed from a second material having a second modulus of elasticity that is lower than the first modulus of elasticity;
securing an exposed electrically conductive functional tip at a distal end of the core; and
securing an insulation layer over a portion of the core.
